(19) United States
(12) Patent Application Publication
(10) Pub. No.: US 2012/0157962 A1
(43) Pub. Date: Jun. 21, 2012

(54) NEEDLE-FREE INJECTOR

(75) Inventors: Nicky CANTON, Shenzhen (CN); Bart HEGEMAN, Alkmaar (NL)

(73) Assignee: EUROPEAN PHARMA GROUP BV, Schiphol - Rijk (NL)

(21) Appl. No.: 12/973,890

(22) Filed: Dec. 20, 2010

(51) Int. Cl. A61M 5/30

(52) U.S. Cl. 604/500; 604/68

(57) ABSTRACT

A needle-free injector is disclosed, the injector comprising a nozzle assembly disposed at a front end of the needle-free injector, wherein the nozzle assembly comprises a piston within a nozzle and adapted to extract and contain a volume of fluid to be injected. The injector also comprises a power house adapted to exert an injection force on the piston to inject the fluid from the nozzle. The injector further comprises a release system, wherein the release system comprises a locking member adapted to move between a locked position and an unlocked position, and a releasing member adapted to move the locking member from the locked position to the unlocked position upon exertion of an unlocking force on the releasing member by a user during operation. While the locking member is in unlocked position, the user can activate the power house by exerting a counter force on the injector. The unlocking force and the counter force are in the same direction as the injection force. A method of releasing a needle-free injector is also disclosed.

### FIELD OF INVENTION

This invention relates to a needle-free injector, and in particular a needle-free injector with a release system for unlocking the injector before injection.

### BACKGROUND OF INVENTION

Some needle-free injectors are provided with release system or unlocking system for preventing premature firing of the injector. For a successful needle free injection, the user is required to operate the release system and position the injector on the injection site perpendicular to the skin before firing, such as by exerting a counter force. The complexity of this combination can easily lead to unsuccessful injections.

### SUMMARY OF INVENTION

In the light of the foregoing background, it is an object of the present invention to provide an alternate needle-free injector with a release system optimized for more convenient injection.

Accordingly, the present invention, in one aspect, is a needle-free injector comprising a nozzle assembly disposed at a front end of the needle-free injector, wherein the nozzle assembly comprises a piston within a nozzle at a back end of the nozzle assembly and adapted to contain a volume of fluid to be injected. The injector also comprises a power house adapted to exert an injection force on the piston to inject the fluid from the nozzle. The injector further comprises a release system, wherein the release system comprises a locking member adapted to move between a locked position and an unlocked position, and a releasing member adapted to move the locking member from the locked position to the unlocked position by exerting an unlocking force on the releasing member by a user during operation. When the injector is unlocked, the user can activate the power house by exerting a counter force on the injector. The unlocking force and the counter force are in the same direction as the injection force.

In an exemplary embodiment of the present invention, the locking member rotates between the locked position and the unlocked position, the releasing member comprises a force translation member translating the unlocking force into rotational force of the locking member causing the locking member to move to the unlocked position.

In another exemplary embodiment, the locked position is offset from the unlocked position by 90 degrees radially.

According to another aspect of the present invention, a method of releasing a needle-free injector is provided. The method comprises the step of providing a power house adapted to exert an injection force to a piston of a nozzle assembly, providing a locking member adapted to move between a locked position and an unlocked position, and exerting an unlocking force on a releasing member in the direction of the injection force, the movement of the releasing member causing the locking member to move from the locked position to the unlocked position. While the locking member is in the unlocked position, exerting a counter force, in the direction of the injection force activates the power house.

There are many advantages to the present invention. The direction of the unlocking force is in line with the counter force required to activate the power house. The unlocking force is operated by the user's thumb, and the counter force is exerted by the user's hand. Both forces being in line ensures an ergonomical positioning of the hand during injection, making it easier for the user to position the injector correctly on the injection site, thus increasing the change of a successful injection.

Another advantage of the present invention is that the release spring ensures that the releasing member must be continuously pressed to keep the locking member at the unlocked position. This eliminates the chance of premature firing due to counter force being exerted accidentally on the nozzle such as dropping the injector onto the ground etc., since the injector is back to the locked position as soon as the pressure on the releasing member is removed.

### BRIEF DESCRIPTION OF FIGURES

FIG. 1a is a cross-sectional view of a needle-free injector at an initial state according to an embodiment of the present invention.

FIG. 1b is a magnified view of a part of the power house of the needle-less injector in FIG. 1a.

FIG. 2a is an exploded diagram in a perspective view of the release system of the needle-less injector in FIG. 1a.

FIG. 2b is a front view of the locking member of FIG. 2a.

FIG. 3 is a cross-sectional view of the needle-free injector when the spring is primed and a fluid is loaded into the injector.

FIG. 4a is a cross-sectional view of the needle-free injector when the releasing member is pressed to unlock the injector for firing.

FIG. 4b is a perspective view of the release system when the injector fires.

### DETAILED DESCRIPTION OF THE PREFERRED EMBODIMENTS

As used herein and in the claims, “comprising” means including the following elements but not excluding others.

As used herein and in the claims, the “forward” direction means substantially along the direction of the injection force, and “backward” or “rearward” direction means substantially opposite to the direction of the injection force.

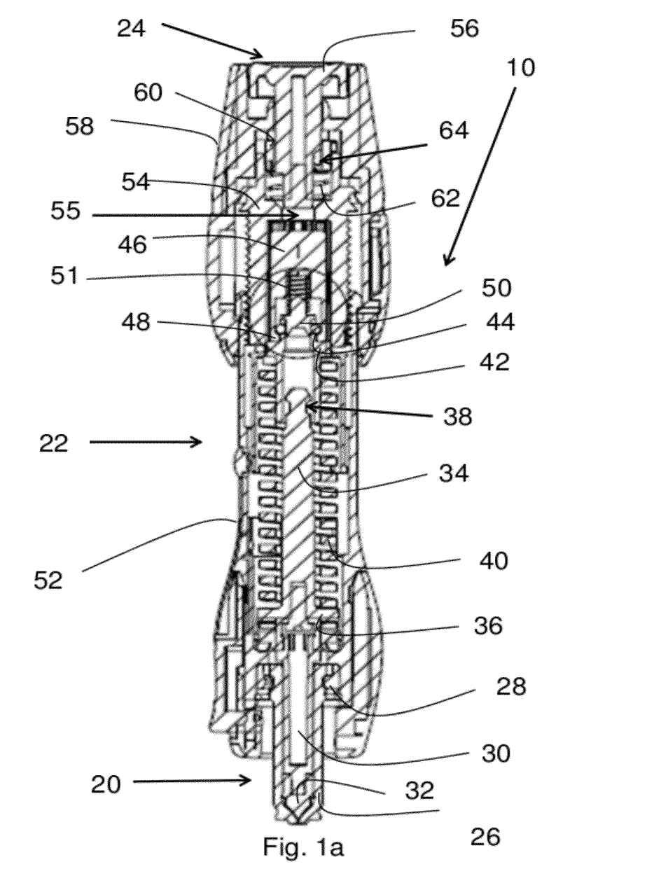

A cross-sectional view of a first embodiment of a needle-free injector 10 at its initial or “released” state is shown in FIG. 1a. The injector comprises a nozzle assembly 20, a power house 22 and a release system 24. The power house 22 is adapted to exert an injection force on the nozzle assembly 20 to release a fluid loaded therein. The release system 24 is adapted to unlock the power house 22 to allow exertion of the injection force.

The nozzle assembly 20 is disposed at the front end of the injector 10, and comprises a nozzle 26 with an injection orifice at a front end thereof. An injector connector 28 is disposed at a peripheral of the rear end of the nozzle assembly 20. A piston 30 and a nozzle ram 32 fit into the bore of the nozzle 26 from its rear end, and are adapted to move forward and backward relative to the nozzle 26. The bore of the nozzle 26 along with the piston 30 and nozzle ram 32 defines an injection chamber having a variable volume.

The power house 22 includes a center shaft 34 disposed at the front end of the power house 22. The center shaft 34 has an enlarged front end 36 adapted to engage with the piston 30 of the nozzle assembly 20. The center shaft 34 is disposed inside the core of a main spring 40, with the back edge of the enlarged front end 36 defining the front stop point of the main spring 40. A locking groove 38 is provided at the rear end of the center shaft 34.

A magnified view of a portion of the power house 22 is shown in FIG. 1b. A sleeve 42 having a flange extending radially from the mid portion. The flange acts as the rear stop point of the main spring 40 (not shown in FIG. 1b). The sleeve 42 allows the center shaft 34 (not shown in FIG. 1b) to protrude therethrough. A plurality of openings are provided towards the rear end of the wall of the sleeve 42, with a ball bearing 44 disposed inside each opening and able to move radially inwards or outwards through the opening. A sleeve cover 50 having a flange extending radially outwards is provided rearwards of the sleeve 42 with the flange engaging with the rear edge of the wall of the sleeve 42. The rear end of the sleeve cover 50 is formed into a rounded bottom. A trigger spring 51 is provided with its front end engaging the sleeve cover 50 and adapted to urge the sleeve cover 50 towards the sleeve 42. The round bottom of the sleeve cover 50 acts as a trigger spring guide.

A retention member 46 having a cavity opened from the front end thereof. This cavity is formed from two cylindrical spaces joined end-to-end, the first cylindrical space disposed at the front end having a larger diameter, and the second cylindrical space with a smaller diameter extending towards the back end of the retention member. The rear end of the trigger spring 51 engages the retention member 46 by extending along the second cylindrical space. The front end of the first cylindrical space has a constricted section forming a neck 48 at its front end, the neck at this initial state is in front of the ball bearings 44.

At this initial state, the sleeve cover 50 blocks the ball bearings from moving radially inwards, while the retention member 46 prevents the ball bearings from moving radially outwards. The neck 48 of the retention member 46 is locked axially relative to the sleeve 42 through the ball bearings 44 such that the retention member 46 will be able to move only when the ball bearings 44 move. This will be explained in more detail in the following paragraphs.

Referring back to FIG. 1a, a hollow cylinder with external threads on its rear end acts as a center body 52 is provided with the center shaft 34 and the main spring 40 inserted within the hollow space therein. The center body 52 is axially fixed to the nozzle assembly 20 at its front portion and threadably connected to a cylinder bush 54 at its rear portion. The cylinder bush 54 has an opening made up of two cylindrical spaces, the first cylindrical space being larger in diameter and adapted to receive the retention member 46, and the second cylindrical space being smaller in diameter and adapted to allow a releasing member to protrude therethrough to access the retention member 46. This will be explained in more detail when the release system 24 is described.

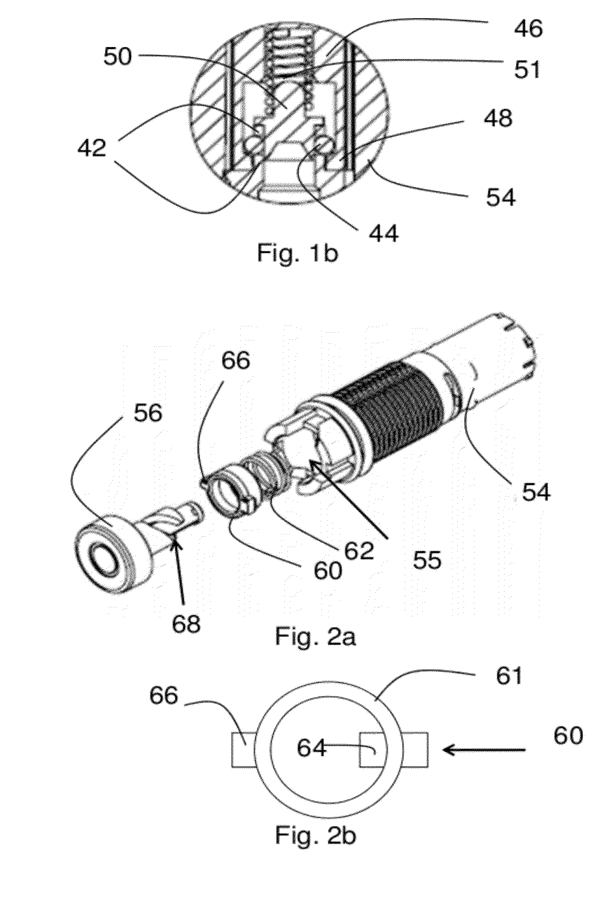

An exploded diagram of the release system 24 according to an embodiment of the present invention is shown in FIGS. 2a and 2b. A releasing member 56 is provided rearward of the second cylindrical space 55 of the cylinder bush 54 adapted to insert through the second cylindrical space 55 to engage with and urge the retention member 46 (not shown in FIGS. 2a and 2b) forward. A locking member 60 is provided at the rear end of the cylinder bush 54, with the outer walls of the cylinder bush 54 and the back body 58 (not shown in FIG. 2) combining to limit the available movement of the locking member 60 to rotation in a predetermined direction only. A release spring 62 is provided between the cylinder bush 54 and the releasing member 56 urging the releasing member 56 away from the cylinder bush 54.

As shown in FIG. 2b, the locking member 60 is a ring-shaped structure 61 with a protrusion 64 extending radially inwards and two extensions 66 extending radially outwards at opposite sides of the ring-shaped structure 61. The releasing member 56, or alternatively known as a release button, has a thick disc-shaped button with a post extending from the front end of the disc-shaped button. A helical groove 68 winds 90 degrees along the exterior of the front portion of the post and is adapted to receive the protrusion 64 of the locking member 60. The rear end of the cylinder bush 54 is provided with corresponding recessions at locations corresponding to the unlocked position, adapted for receiving the extensions 66.

In the operation of the device, first the main spring 40 needs to be primed for injection. This is achieved by rotating the back body 58 relative to the center body 52 in a predetermined direction (e.g. clockwise when looking from the rear end). The rotational motion of the back body 58 drives the cylinder bush 54 into rotation as it is radially locked to the back body 58. As the cylinder bush 54 is threadably connected to the center body 52, this rotation will cause the cylinder bush 54 to move towards the front end of the center body 52. The cylinder bush 54 also urges the retention member 46, the trigger spring 51, the sleeve cover 50, the sleeve 42 and the ball bearings 44 to move forward towards the center shaft 34, while the center shaft 34 remained stationary relative to the nozzle assembly 20. Since the rear end point of the main spring 40 (i.e. the sleeve 42) moves towards the front end point of the main spring 40 (i.e. the enlarged end 36 of the center shaft 34), the main spring 40 is compressed with this movement. As this compression continues, the center shaft 34 starts to engage and urge the sleeve cover 50 rearwards towards the retention member 46, compressing the trigger spring 51 in the process.

When the ball bearings 44 are level to the locking groove 38 of the center shaft 34, the ball bearings 44 move radially inwards to engage with the locking groove 38 of the center shaft 34. At the same time, the retention member 46 is urged rearwards with respect to the sleeve 42 by the trigger spring 51. When the neck 48 of the retention member 46 is level to the ball bearings 44 and the locking groove 38, the ball bearings 44 are locked within the locking groove 38 as the neck 48 of the retention member 46 prevents the ball bearings 44 from moving radially outwards. At this point, the center shaft 34 is locked to the retention member 46, therefore keeping the main spring 40 at a compressed or primed state. The injector is said to be triggered at this stage.

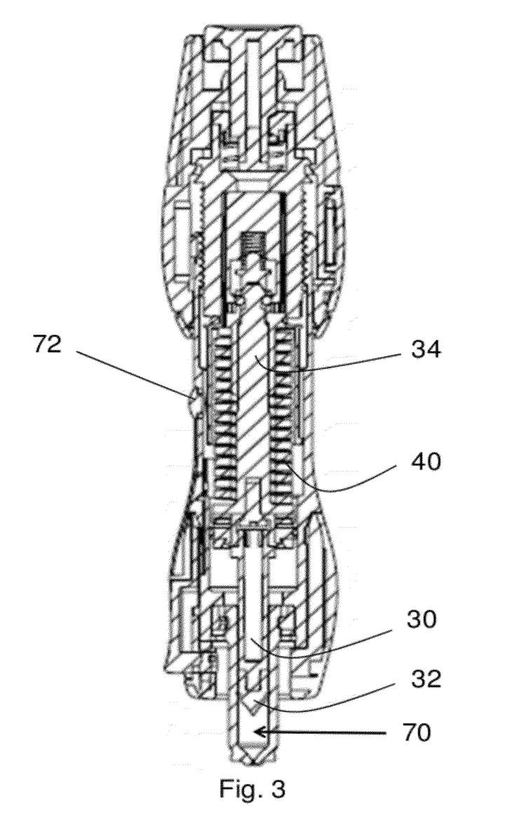

Afterwards, a fluid to be injected is loaded into the injector 10. A fluid source (not shown) is attached to the nozzle 26 with appropriate adaptors to allow fluid flow therebetween. The back body 58 is then rotated with respect to the center body 52 in a direction opposite to the predetermined direction in the previous stage (e.g. counterclockwise when looking from the rear end). As such, the back body 58 will move rearward due to this rotation. The center shaft 34 also moves rearwards and is locked to the retention member 46 through the ball bearings 44. The piston 30 of the nozzle assembly 20 also moves rearwards to enlarge the injection chamber 70 as shown in FIG. 3. When the injection chamber 70 is enlarged, fluid is extracted or drawn from the fluid source into the injection chamber 70 as a result.

A current volume or dosage of the fluid loaded can be monitored through a lens 72 disposed on the center body 52. The sleeve 42 (not shown in FIG. 3) displays the dosage information while rotating with respect to the center body 52, for example from 0 to 50 units. This information is magnified by the lens 72 to enlarge the displayed information for ease of reading. The injector after loading 30 units of fluid is shown in FIG. 3.

The injector as disclosed in the present embodiment is activated or fired when a counter force is exerted on the injector 10, or more specifically the back body 58. When a user pushes the injector 10 forward while the nozzle head is in contact with an object, for example a patient's skin, this forward force counter to the force exerted on the nozzle head by the object will result in the retention member 46 moving forward, causing the ball bearings 44 to move radially outwards from the locking groove 38 of the center shaft 34 and thus freeing the center shaft 34 to move forward. The center shaft 34 then pushes the piston 30 towards the injection orifice, releasing the fluid through the injection orifice. The following paragraphs will explain how the release system 24 works to prevent unintended firing of the injector.

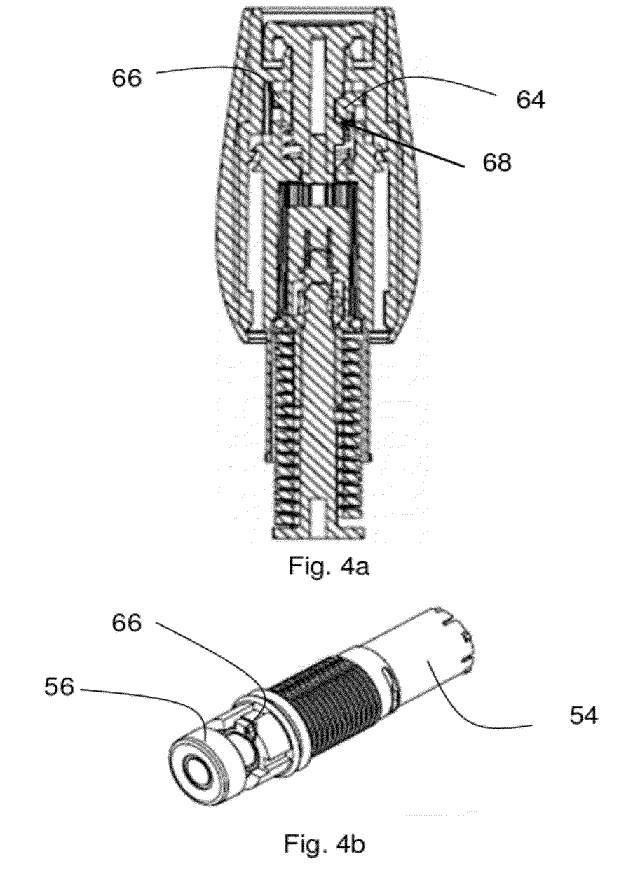

Referring to FIGS. 4a and 4b, when the release button is depressed, the inner edge of the helical groove 68 and the protrusion 64 translate the axial motion or axial force of the release button into the rotational motion or rotation force of the locking member 60 as the helical groove 68 is adapted to be tilted at an angle with respect to the axial motion. More specifically, while the release button is moving forward due to the button being depressed, the protrusion 64 will slide along the tilted inner edge of the helical groove 68, therefore causing the angular position of the protrusion 64 to change correspondingly, thus leading to the rotation of the locking member 60. Consequently, the rotational motion of the locking member 60 causes the locking member 60 to move to the unlocked position. At the unlocked position, the protrusion 64 of the locking member 60 is urged to the rear end of the helical groove 68. The extensions 66 are aligned with corresponding recessions of the cylinder bush 54, thus allowing the release button to move further forward and pushing the extensions 66 into the corresponding recessions upon exertion of a forward force on the back body 58. The release system 24, when the extensions 66 are inserted in the recessions of the cylinder bush 54, is shown in FIG. 4b (back body is not shown). The forward movement of the back body 58 also moves the release button when the locking member 60 is at the unlocked position, and the release button, through the second cylindrical space 55 in the cylinder bush 54, urges the retention member 46 forward. The injector will then fire as explained above.

Once pressure is removed from the release button, the release spring 62 will urge the release button rearwards, causing the locking member 60 to move back to the locked position. Therefore, the injector will only fire when counter force is exerted on the nozzle 26 while pressing the release button continuously.

In another embodiment, the release spring 62 is disposed between the back body 58 and the release button.

In one embodiment, the locked position is offset by 90 degrees from the unlocked position. In other embodiments, the offset can be between 30 to 90 degrees, as long as substantial pressure needs to be exerted on the release button to move the locking 230 member 60 from the locked position to the unlocked position. The helical groove 68 therefore has a radial length of 90 degrees with respect to the transverse plane.

In one embodiment, the helical groove 68 makes an angle of around 45 degrees with the axial direction. In general, the larger the angle is, the harder the translation of force is but the total length of the groove and thus the releasing member 56 is reduced. In other embodiments, the angle can be between 30 to 60 degrees.

In an exemplary embodiment, the releasing member 56 is provided at a radial center of the injector such that the unlocking force is aligned to the injection force at the same axis.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, the locking member 60 does not need to be in the shape of a ring, and does not need to be in one single piece. The locking member 60 also does not need to be rotatable and can move in another direction, such as radially outward or inward, axial or in other arbitrary directions. For example, the locking member 60 can be in two separate pieces that move radially outward from the locked position to the unlocked position when the releasing member 56 moves forward.

The releasing member 56 can alternatively be disposed on the radial peripheral of the injector other than at the back end, as long as the releasing member 56 is able to move forward to unlock the injector for firing.

Any number of protrusions 64 and extensions 66 can be implemented for the locking member 60. The number of corresponding helical grooves 68 in the releasing member 56 is also not limited. For example, in another embodiment, there are two protrusions 64 at opposite sides of the ring-shaped structure 61 aligned with the extensions 66, and two helical grooves 68 are provided at the releasing member 56 adapted to receive the two protrusions 64.

What is claimed is:

1. A needle-free injector comprising
a) a nozzle assembly disposed at a front end of said needle-free injector, said nozzle assembly comprises a piston within a nozzle adapted to extract and contain a volume of fluid to be injected;
b) a power house adapted to exert an injection force on said piston to inject said fluid from said nozzle;
c) a release system, wherein said release system comprises
i) a locking member adapted to move between a locked position and an unlocked position;
ii) a releasing member adapted to move said locking member from said locked position upon exertion of an unlocking force on said releasing member by a user during operation;
wherein when the injector is unlocked, said user can activate said power house by exerting a counter force on said injector, said unlocking force and said counter force are in the same direction as the direction of said injection force.

2. The needle-free injector according to claim 1, wherein said injection force is exerted on said piston upon exertion of a counter pressure on said nozzle, the direction of said counter pressure being in an opposite direction to said unlocking force.

3. The needle-free injector according to claim 1, wherein said locking member rotates between said locked position and said unlocked position; said releasing member comprises a force translation member translating said unlocking force into rotational force of said locking member causing said locking member to move to said unlocked position.

4. The needle-free injector according to claim 3, wherein said locking member comprises at least one extension extending from a ring-shaped structure; said force translation member being at least one helical groove having an inner edge tilted relative to said direction of unlocking force, said helical groove coupling with at least one said extension, such that said extension moves along said helical groove during forward movement of said releasing member, causing said locking member to rotate to said unlocked position.

5. The needle-free injector according to claim 4, wherein said helical groove forms an angle of around 45 degrees with respect to the direction of said unlocking force.

6. The needle-free injector according to claim 3, wherein said locked position is offset from said unlocked position by 90 degrees radially.

7. The needle-free injector according to claim 1, wherein said release system further comprises a release spring urging said releasing member towards said locked position, such that said releasing member is at said locked position when said unlocking force is not exerted, and when said releasing member moves said locking member from said locked position to said unlocked position, a continuous pressure is needed to keep said locking member at said unlocked position.

8. A method of releasing a needle-free injector for activation, comprising the steps of:
a) providing a power house adapted to exert an injection force to a piston of a nozzle assembly;
b) providing a locking member adapted to move between a locked position and a unlocked position;
c) exerting an unlocking force on a releasing member in the direction of said injection force, the movement of said releasing member causing said locking member to move from said locked position to said unlocked position;
whereby while the locking member is in said unlocked position, exerting a counter force, in the direction of the injection force activates said power house.

9. The method according to claim 8, wherein said locking member rotates from said locked position to said unlocked position, wherein a portion of said unlocking force is translated into a rotational force causing said rotation of said locking member.

10. The method according to claim 9, wherein said locking member comprises a protrusion received in a helical groove of said releasing member; said helical groove translates said unlocking force exerted on said releasing member into said rotational force of said protrusion of said locking member.
